SEQUENCE LISTING (58 sequences)

SEQ ID NO: 1 - DNA, 40 bp, Homo sapiens
ggtcttacataagaggactcagaaaatactttgtgatgat

SEQ ID NO: 2 - DNA, 40 bp, Homo sapiens
aactccttttgagagcgcagtgttcggtgcaggtccccag

SEQ ID NO: 3 - DNA, 40 bp, Homo sapiens
atcatcacaaagtattttctgagtcctcttatgtaagacc

SEQ ID NO: 4 - DNA, 40 bp, Homo sapiens
tgaggtgagaggatctcttgagcacagatgatgggatagg

SEQ ID NO: 5 - DNA, 40 bp, Homo sapiens
aaacgtccacttgcagattttatgtaattggaagttgggg

SEQ ID NO: 6 - DNA, 40 bp, Homo sapiens
agcaggaaaatatatgccccaccttccctttctctgaccc

SEQ ID NO: 7 - DNA, 40 bp, Homo sapiens
aggaagactgcctactcccacaggcctgaaagcgctccaa

SEQ ID NO: 8 - DNA, 40 bp, Homo sapiens
agcattcgggccagggttcactcaggctcagggcacattg

SEQ ID NO: 9 - DNA, 40 bp, Homo sapiens
gcatttggtggtctataagcacacccgcccacaccaatct

SEQ ID NO: 10 - DNA, 40 bp, Homo sapiens
caagaccagcctgaggatgactgtctcttagaggtggaga

SEQ ID NO: 11 - DNA, 40 bp, Homo sapiens
cactccttttgagagcgcagtgttcaggtgcagggtcccc

SEQ ID NO: 12 - DNA, 40 bp, Homo sapiens; identical to SEQ ID NO: 3

SEQ ID NO: 13 - DNA, 40 bp, Homo sapiens
aacagaaagattcgtccccaaatccaatctgtcttccatc

SEQ ID NO: 14 - DNA, 40 bp, Homo sapiens
agcaggaaaatatatgccccaccttccctttctctgccct

SEQ ID NO: 15 - DNA, 40 bp, Homo sapiens; identical to SEQ ID NO: 1

SEQ ID NO: 16 - DNA, 40 bp, Homo sapiens; identical to SEQ ID NO: 9

SEQ ID NO: 17 - DNA, 40 bp, Homo sapiens
atcatcctgggctttctgcacttcccataggtaatcaaag

SEQ ID NO: 18 - DNA, 40 bp, Homo sapiens; identical to SEQ ID NO: 8

SEQ ID NO: 19 - DNA, 100 bp, Homo sapiens
atgaagctatttataataaaacaaactttattaaatctagtttaaatgccttactctcttttttgccttctgacttctttccttctattcgagatctcct

SEQ ID NO: 20 - DNA, 100 bp, Homo sapiens
tttcattgttgctgtttttcaaattgattttgggatccagcctgttattctactcccttaacttcatgggatatgtaattggaagttggggtactttacc

SEQ ID NO: 21 - DNA, 100 bp, Homo sapiens
tctccctttagacttcaaacacttcaaaatatgacttcactacaaagctttatagaatgccagccttccacagagtatgtaaataatgcctagttttgaa

SEQ ID NO: 22 - DNA, 100 bp, Homo sapiens
ccagcacatttgtctataaatttacattcttggatattagcaaaattgcaaacagaccaatttatgcctacagcctcctagtacaaagacctttaaccta

SEQ ID NO: 23 - DNA, 100 bp, Homo sapiens
tccagtgtttgtgggttgagcagtattattgcatggcccagtggtggtggttgatgttcctggaagtagaggacaaacgggcaacataccttggtagtcc

SEQ ID NO: 24 - DNA, 100 bp, Homo sapiens
tgcaagtggttgcagttcttttgctttgccaccaccactgggccatgcaaaacctgcacgattcctgctcaaggaacctctatgtttccctcttgttgct

SEQ ID NO: 25 - DNA, 100 bp, Homo sapiens
caggaggaggtgatggacccactgggtggtgaagaacagtttctcttccaaaattacttcccacccaggtggccagattcatcaactcaccccaacacag

SEQ ID NO: 26 - DNA, 100 bp, Homo sapiens
atctgtaaaattgggatcatcacactttccttttattggggtttaaatgaatacccaaagacaaaagaaaattggtaatagaggtaaaaagggactcaag

SEQ ID NO: 27 - DNA, 100 bp, Homo sapiens
tggccgaggccatcttctaaataaatgtgtggaagagaaactgttcttcagtatttggtgtcttttggagtgtggattcgcactcctcccgcttacagac

SEQ ID NO: 28 - DNA, 100 bp, Homo sapiens
aggacgggtgcccgggtccccagtccctccgccacgtgggaagcgcggtccagaccaatttatgcctacagcctcctagtacaaagacctttaacctaat

SEQ ID NO: 29 - DNA, 100 bp, Homo sapiens; identical to SEQ ID NO: 19

SEQ ID NO: 30 - DNA, 100 bp, Homo sapiens; identical to SEQ ID NO: 25

SEQ ID NO: 31 - DNA, 100 bp, Homo sapiens; identical to SEQ ID NO: 23

SEQ ID NO: 32 - DNA, 100 bp, Homo sapiens
gtccgttggtggtgaactgggcaagataattgcatggcccagtggtggtggttgatgttcctggaagtagaggacaaacgggcaacataccttggtagtc

SEQ ID NO: 33 - DNA, 100 bp, Homo sapiens
agattggtctataattttcttttactatcttcagtatttggtatctttgggagtgtggattcgcactcctcccgcttacagaccaccaaatgcccctatc

SEQ ID NO: 34 - DNA, 100 bp, Homo sapiens; identical to SEQ ID NO: 20

SEQ ID NO: 35 - DNA, 100 bp, Homo sapiens; identical to SEQ ID NO: 27

SEQ ID NO: 36 - DNA, 100 bp, Homo sapiens; identical to SEQ ID NO: 24

SEQ ID NO: 37 - DNA, 100 bp, Homo sapiens
gtaagccattgtggctttcctgaccagcccaccaccactgggccatgcaaaacctgcacgattcctgctcaaggaacctctatgtttccctcttgttgct

SEQ ID NO: 38 - DNA, 100 bp, Homo sapiens; identical to SEQ ID NO: 22

SEQ ID NO: 39 - DNA, 100 bp, Homo sapiens
ggaaatggagccaggcgctcctgctggccgcgcaccgggcgcctcacaccagaacatcgcatcaggactcctaggacccctgctcgtgttacaggcgggg

SEQ ID NO: 40 - DNA, 100 bp, Homo sapiens
tcaagcagaaaaaccatgaagatttaaaaacttgtaaatatttgaatgtgggctccaccccaacagtcccccgtggggaggggtgaaccctggcccgaat

SEQ ID NO: 41 - DNA, 100 bp, Homo sapiens
ctaagggacactacaggaaaccagccccgaagtgatttcttttgaaattccaaatctttctgtccccaatcccctgggattcttccccgatcatcagttg

SEQ ID NO: 42 - DNA, 100 bp, Homo sapiens
cctcgaagcctgtgccaacctagcccattcctcaggctcagggcctcctcacatctgtgccagcagctcctcctcctgcctccaccaatcggcagtcagg

SEQ ID NO: 43 - DNA, 100 bp, Homo sapiens
cattgttactgtgatatgctataattattctcaccttatgtgtccaaggaatactaacattgagattcccgagattgagatcttctgcgacgcggcgatt

SEQ ID NO: 44 - DNA, 100 bp, Homo sapiens
atggagaatacagcacattattaggagtaagtttccttaaacacattttgattttttgtacaatatgttcctgtggcaatgtgccccaactcccaattac

SEQ ID NO: 45 - DNA, 100 bp, Homo sapiens
tttgccaccttcctgccactttgtagatgcaagatcttgggcaagttcccgtgggcgttcacggtggtttccatgcgacgtgcagaggtgaagcgaagtg

SEQ ID NO: 46 - DNA, 100 bp, Homo sapiens
cagtggaaacaaagccactgggaagttcaaactgagagaagcccaccacaagtctagactctgtggtattgtgaggatttttgtcaacaagaaaaacccc

SEQ ID NO: 47 - DNA, 100 bp, Homo sapiens
agtatatcatcagttatttttcaaggttttctaagtaaacagtttctcaacctttaccccgttgctcggcaacggcctggtctgtgccaagtgtttgctg

SEQ ID NO: 48 - DNA, 100 bp, Homo sapiens
tcagggaggggatgttgactgcattttggaggttcagggcctactaacaactgtgccagcagctcctcctcctgcctccaccaatcggcagtcaggaagg

SEQ ID NO: 49 - DNA, 100 bp, Homo sapiens; identical to SEQ ID NO: 39

SEQ ID NO: 50 - DNA, 100 bp, Homo sapiens; identical to SEQ ID NO: 40

SEQ ID NO: 51 - DNA, 100 bp, Homo sapiens
cccgggaccgaccccaggaagagccaggggcccgggtgatccctgcgggggtctggctttcagttatatggatgatgtggtattgggggccaagtctgta

SEQ ID NO: 52 - DNA, 100 bp, Homo sapiens
aatgaaaatctcattgatttttcacttataggttttaccttagagctcctcctctgcctaatcatctcatgttcatgtcctactgttcaagcctccaagc

SEQ ID NO: 53 - DNA, 100 bp, Homo sapiens
agaattgatacctaagctgagcagaaatgaggccgaccatgaagtgagtgcctaatcatctcatgttcatgtcctactgttcaagcctccaagctgtgcc

SEQ ID NO: 54 - DNA, 100 bp, Homo sapiens
cgtaggaaagacaaggtggcattgatggaaagcagtagtttttgagcccttcgcagacgaaggtctcaatcgccgcgtcgcagaagatctcaatctcggg

SEQ ID NO: 55 - DNA, 100 bp, Homo sapiens
ttaaaaaggagttttgtttgttagtctattcactcatttcaaggaacatagaagaagaactccctcgcctcgcagacgaaggtctcaatcgccgcgtcgc

SEQ ID NO: 56 - DNA, 100 bp, Homo sapiens
cagttccctggctccaagctccctcaaaagatgcccagctggcctttcccaaaggccttgtaagttggcgagaaagtaaaagcctgttttgcttgtatac

SEQ ID NO: 57 - DNA, 100 bp, Homo sapiens
acatgcccttcacttcagcctgatgctcctggcataagctcagcaattttggagtgcgaatccacactccaaaagacaccaaatattcaagaacagtttc

SEQ ID NO: 58 - DNA, 100 bp, Homo sapiens
aatttcccctgaatagctgcagtactcacagacacactggatgctactcacctctgcctaatcatctcatgttcatgtcctactgttcaagcctccaagc

(19) United States
(12) Patent Application Publication
(10) Pub. No.: US 2015/0119260 A1
(43) Pub. Date: Apr. 30, 2015

(54) CIRCULATING CANCER BIOMARKER AND ITS USE

(71) Applicant: NATIONAL TAIWAN UNIVERSITY, TAIPEI (TW)

(72) Inventors: PEI-JER CHEN, Taipei (TW); SHIOU-HWEI YEH, Taipei (TW); CHIAO-LING LI, Taipei (TW); DING-SHINN CHEN, Taipei (TW)

(21) Appl. No.: 14/515,550

(22) Filed: Oct. 16, 2014

Related U.S. Application Data
(60) Provisional application No. 61/892,796, filed on Oct. 18, 2013.

(51) Int. Cl. C12Q 1/68; C12Q 1/70

(52) U.S. Cl. CPC C12Q 1/6886; C12Q 1/706; C12Q 2600/156; C12Q 2600/112
USPC 506/2; 435/5; 506/9; 536/23.1

(57) ABSTRACT

The present invention provides a chimera nucleic acid obtained from circulatory system for monitoring tumor status. The nucleic acid comprises partial sequence derived from host genome and partial sequence derived from non-host genome. The partial sequence derived from host genome and the partial sequence derived from non-host genome form a chimera junction. The chimera junction is obtained from cell-free nucleic acids and is indicative of disease status.

### REFERENCE TO AN ELECTRONIC SEQUENCE LISTING

The contents of the electronic sequence listing (US57470_ST25.txt; Size: 11.7 KB; and Date of Creation: Nov. 25, 2014) is herein incorporated by reference in its entirety.

### CROSS REFERENCE TO RELATED APPLICATION

This application claims the benefit of U.S. Provisional Application No. 61/892,796, filed Oct. 18, 2013, the contents of which are adopted herein by reference.

### FIELD

Aspects of the present disclosure relate generally to the field of using circulating nucleic acids in a subject as a biomarker to identify and monitor a disease development in the subject.

### BACKGROUND

The fundamental cause of tumor/cancer has been attributed to genetic alterations caused by hereditary or environmental factors. These genetic alterations, once ir-repaired or irreparable, will accumulate and eventually cause normal cells to become malignant. As a tumor/cancer develops its own unique spectrum of genetic alterations, monitoring these alterations can provide information about the tumor/cancer.

Both normal and tumor/cancer cells undergo cycles of turnover where chromosomes of dead cells are fragmented and released into body fluids, such as blood circulation. Sequencing of these fragments indicates that these circulating cell-free DNA from the blood or serum of cancer patients carry the genetic alterations from the original tumor/cancer. This finding points to the potential of using circulating cell-free DNA.

The conventional design of using host genome sequences containing specific genetic alterations as probes for capturing cancer/tumor-specific nucleic acid sequences from total circulating cell-free DNA works for advanced cancer, where the tumor is sufficiently large and a significant amount of tumor-specific nucleic acid sequences (more than 5% of total circulating DNAs) is released into the circulation. Given its limited amount (0.01%-1% in total blood), circulating cell-free DNA is hard to detect even in an advanced cancer. As a result, for early or intermediate stage of cancer, the proportion of circulating cancer/tumor DNA is too low to be reliably detected. Moreover, cancer/tumor-specific mutations are usually single-base mutations, small insertions or deletions which are very difficult to be separated from nucleic acid sequences without such mutations released from the non-tumor somatic cells. In other words, not all circulating DNA bears the altered genetic information; most of the circulating DNA is unaltered and from host genome.

### BRIEF DESCRIPTION OF THE DRAWINGS

Many aspects of the present disclosure can be better understood with reference to the following figures. The components in the figures are not necessarily drawn to scale with the emphasis instead being placed upon clearly illustrating the principles of the present disclosure.

FIG. 1 schematically shows general progression of virus-infected cells.

FIG. 2 schematically shows an exemplary method of obtaining target circulating cell-free DNA.

FIG. 3 shows the specificity of viral-host junction.

FIG. 4 shows the changes in the amount of specific viral-host junction before and after tumor resection.

### DETAILED DESCRIPTION

1. Introduction

Certain human tumors/cancers, such as hepatocellular carcinoma (HCC), are caused by chronic infection of hepatitis B virus (HBV). These cancers accumulate genetic alterations in their genomes. Among such alterations, a unique one is the integration of viral genome into the host genome, usually occurring in the early stage of infections. Superimposed upon these mutations are other somatic mutations that continue to occur and finally transform the cells to tumor/cancer.

As noted, when HCC cells turn over, fragmented genetic contents will be released into the body fluids. Circulating DNA which is DNA that floats freely in the circulatory system, such as blood, usually comprises DNA fragments. These fragments include those from host genome, from viral genome, and/or from the viral integration sites, such as the viral-host junction.

Infected cells, such as hepatocytes in a HBV-infected patient, proliferate if they become cancerous and so is the amount of the viral integrants carried by the infected cells. The amount of viral integrants thus is in proportion to the size of tumor/cancer in general. In addition, as the viral DNA integrates into the host genome at different sites, each tumor/cancer carries a unique spectrum of viral integration sites. This observation indicates that the viral integration sites, and/or the viral-host junction, are cancer/tumor-specific and can be used as biomarkers for the diagnosis of cancer/tumor development.

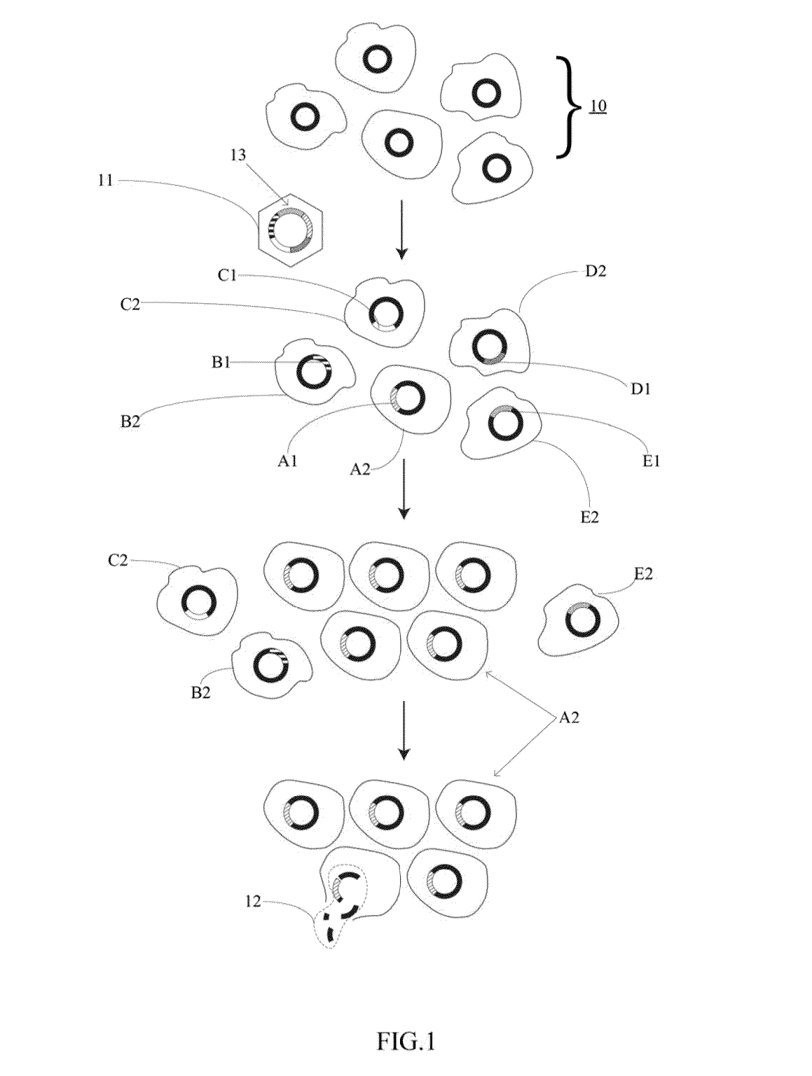

FIG. 1 shows a cancer development process of cells. Referring to FIG. 1, hepatocytes 10 in a subject generally have the same host genome. Referring again to FIG. 1, hepatocytes 10 comprise a plurality of hepatocytes A2, B2, C2, D2 and E2. Upon HBV 11 infection, HBV 11 can integrate its viral genome 13 into the host genome of the infected hepatocytes. Parts of HBV genome 13 are integrated into the host genome, generating infected hepatocytes with different viral integration sites and different integrated viral gene sequence. As show in FIG. 1, viral sequence A1 is integrated into cell A2, viral sequence B1 is integrated into cell B2, viral sequence C1 is integrated into cell C2, viral sequence D1 is integrated into cell D2 and viral sequence E1 is integrated into cell E2. The integration of HBV DNA sequences creates viral-host junctions in host cell genome. Infected cells A2, B2, C2, D2 and E2 grow, develop and accumulate additional genetic alterations with time. Both host and viral sequences, altered or not, might lead to proliferation, stable stage or cell death. Referring again to FIG. 1, cell A2 carries the alterations that induce malignant transformation and lead to proliferation or clonal expansion. It is to be noted that a viral-host junction may lead to malignant transformation or may be an insignificant integration that does not lead to proliferation.

Referring again to FIG. 1, infected cell A2 proliferates, expands in cell number and transforms into a malignant cell, which subsequently forms a cancerous or tumorous cell cluster. Cells B2, C2, D2, E2 do not go through malignant progression and remain in very small population or die out. All infected cells A2 bear the same hereditary information, including the host genome, at least partial viral genome, and the viral-host junctions. If the infected cells A2 proliferate, the number of cell A2-specific viral-host junctions would increase proportionally in general. The same viral-host junctions are present in the same cancerous cell lineage whether they trigger cancer development or not. As depicted schematically in FIG. 1, the cancerous clone goes through rapid proliferation and turnover, and some of the infected cells A2 rupture and die. DNA strands 12 of these ruptured and dead infected cells A2 are released into the circulatory system or body fluids such as blood. These DNA strands 12 become fragmented, float freely through the circulatory system and become part of circulating cell-free DNA (ctDNA) in the blood stream. As used herein, with reference to the present application, it shall be clearly understood that the terms “circulating cell-free DNA”, “circulatory cell-free DNA” and “ctDNA” refer to DNA that is obtained from the blood stream or circulatory system of a subject or patient, wherein the DNA that is obtained from the blood stream or circulatory system of the subject or patient is either substantially free of other cellular components, or is essentially entirely free of other cellular components. Some of the ctDNA is later on digested or cleaned by functional cells such as macrophages while some remain in the blood stream especially when the ctDNA is in large amount. By examining and/or detecting the ctDNA in the circulatory system or body fluids, one can obtain information about cancer/tumor development.

2. Methods

Methods of performing the present invention are described below. It is to be noted that the methods, material and process described below are exemplary embodiments, and do not limit the scope of the invention in any way.

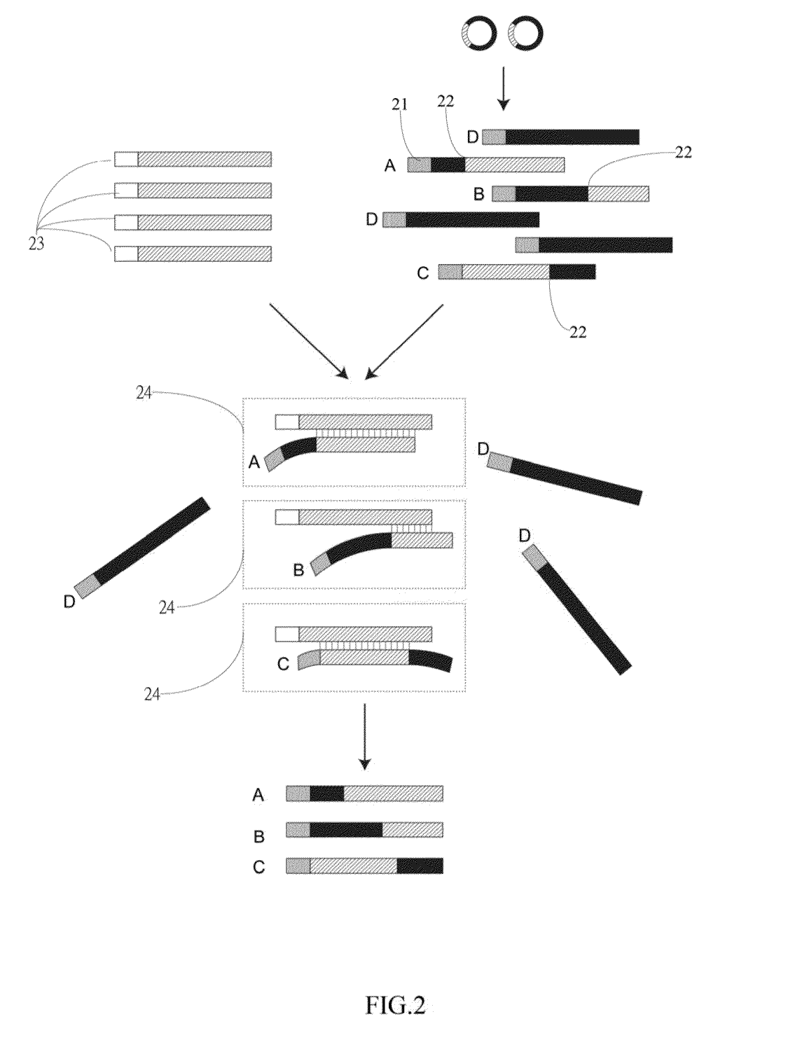

Referring to FIG. 2, a schematic view of isolating target ctDNA is shown. Circulating DNA from dead tumor/cancer cells is released into blood in fragments. Such ctDNA is collected and ligated with adaptors 21 and forms ctDNA A, B, C and D. The ctDNA is amplified by using any suitable approach, for instance, using a primer complementary to the sequence of the adaptor 21 in an appropriate amount. It is to be noted that preferred amplification methods amplify all ctDNA in a similar or the same proportions so that the amplified ctDNA provides genuine information as to the amount of ctDNA existing in the blood. In FIG. 2, sequences derived from viral genome are designated in hatch area while sequences derived from host genome are designated in black. The amplified ctDNA can be categorized into ctDNA having only host genome sequences (ctDNA D), ctDNAs having only viral genome sequence (not shown), and ctDNA having both viral and host genome sequences and thus comprising viral-host junctions 22 (ctDNA A, B, and C). According to a preferred approach, all ctDNA is incubated with polynucleotide probes 23 (derived from the viral genome sequence) to allow hybridization to occur. It is to be noted that the probes 23 shown in FIG. 2 may have different sequences even though all drawn alike. Referring again to FIG. 2, only ctDNA having viral genome sequence alone and ctDNA having at least a viral-host junction (ctDNA A, B and C) would form probe-ctDNA complexes 24. These complexes are isolated from the ctDNA that does not hybridize with the probe. The target ctDNA, the ctDNA having only viral genome sequence and ctDNA having at least a viral-host junction are then obtained from the complexes and separated. The sequences of target ctDNA are obtained. Tissue origins of the target ctDNA are identified based on tissue tropism and specificity of virus infection.

2.1 Subjects

Human subjects are employed in the tests to illustrate the present invention. Subject 1 has a 12×10×9 (cm) tumor diagnosed by computer tomography. According to the histological report when Subject 1 is employed in this test, Subject 1 is defined as a Grade III HCC patient. Subject 2 has a 18×13.5×9 (cm) tumor diagnosed by computer tomography. According to the histological report, Subject 2 is defined as a Grade III HCC patient. Subject 3 has s 8×7.5×7 (cm) tumor identified by computed tomography. According to the histological report, Subject 3 is defined as a Grade III HCC patient. Subject 4 has a 2×2×2 (cm) tumor and is at Grade II. Subject 5 has a tumor smaller than 2×2×2 (cm) and the stage of the cancer development is not determined and/or not available at the time of test enrollment.

2.2 Obtaining ctDNA in Subjects

Multiple blood samples are obtained from each subject. Each time, blood is drawn, collected in a clinically suitable container and, if needed, stored in a suitable condition for later analysis. Each blood sample is processed to obtain serum, such as by centrifugation. ctDNA is extracted by a commercial kit, for example, MagNA Pure LC Total Nucleic acid Isolation kit (Roche). The tumor tissues are obtained and genomic DNAs of tumor cells are extracted.

2.3 Providing Probes

Polynucleotides having HBV genome sequence are used as probes here. The probes can be either synthesized or obtained from the fragmentation of viral genome. Synthesis of the probes is described. Information of whole HBV genome sequences is obtained from the National Center for Biotechnology Information. Polynucleotides are synthesized according to the HBV genome sequence and cover the whole HBV genome sequence. The polynucleotides are synthesized using commercial kit, for example, Ion TargetSeq Custom Enrichment Kit (Life Technologies). All polynucleotides are about 50 to 200 or 50 to 120 residues in length. After the synthesis of probes, each probe is labeled, for example biotinylated, at least one end of the polynucleotide. Biotinylation of probes can be performed by using commercial kit, for example, Ion TargetSeq Custom Enrichment Kit (Life Technologies). The probes are subsequently attached or linked to a bead, for example through biotin.

2.4 Ligating Adaptors

In order to proportionally amplify the ctDNAs obtained from the subject, certain DNA with known sequences are attached or ligated to at least one end or both ends of the ctDNA. Ligating adaptors to at least one end or both ends of the ctDNA can be performed by using TruSeq DNA Sample Preparation (Illumina), IonTorrent (Life Technologies), or other equivalent reagents.

2.5 Amplifying Target ctDNAs

After the ctDNAs are ligated with adaptors, each ctDNA in the sample from the subject is amplified, for example by using TruSeq DNA Sample Preparation (Illumina) or IonTorrent (Life Technologies).

2.6 Capturing and Isolating Target ctDNAs

ctDNA samples of subjects are mixed with beads coated with biotinylated probes and incubated to allow hybridization between ctDNA and the probes. The ctDNA that have at least partial viral sequences anneal to the complementary sequences on the probes and form a bead-probe-ctDNA complex. The ctDNA that does not bind to the probes float freely and does not form any complex. The bead-probe-ctDNA complexes are separated from non-binding ctDNA by, for example, centrifugation. The complexes are obtained and target ctDNA is removed from the complexes and collected. Capturing of circulating DNA hybridized with the probes can be performed by using TargetSeq Hybridization & Wash Buffer Kit (Life Technologies), or by other equivalent reagents.

2.7 Sequencing and Identifying Target ctDNA

Primers having complementary sequences to the adaptor sequences are used to sequence the target ctDNAs. Target ctDNA is sequenced using IonTorrent platform, HiSeq 2500 (Illumina), or some other sequencing platforms.

3. Results

3.1 Target ctDNA Sequences

Subject 1

Table 1 shows top ten target sequences identified in the DNA samples obtained from Subject 1 tumor tissue. As shown, a junction sequence is inserted into the host chromosome (Host Chromosome #) at a specific integration position (Integration Position) with an accumulated read number (Accumulated Reads). Accumulated read number is obtained by sequencing result. Sequences having the same junction are counted to give the number of the junction present in the sample. Each sequence contains at least partial viral genome sequence (underlined) and partial host genome sequence and forms a viral-host junction.

TABLE 1
Junction Data of Subject 1 Tumor

| | Host | | | Accumu- |
|---|---|---|---|---|
| SEQ ID | Chromosome | Integration | Junction | lated |
| NO: | # | Position | Sequence | Reads |
| 1 | 17 | 22247083 | GGTCTTACATA | 290 |
| | | | AGAGGACTCAG | |
| | | | AAAATACTTTG | |
| | | | TGATGAT | |
| 2 | 17 | 22251295 | AACTCCTTTTG | 234 |
| | | | AGAGCGCAGTG | |
| | | | TTCGGTGCAGG | |
| | | | TCCCCAG | |
| 3 | 1 | 121360041 | ATCATCACAAA | 192 |
| | | | GTATTTTCTGA | |
| | | | GTCCTCTTATG | |
| | | | TAAGACC | |
| 4 | 12 | 118876274 | TGAGGTGAGAG | 115 |
| | | | GATCTCTTGAG | |
| | | | CACAGATGATG | |
| | | | GGATAGG | |
| 5 | X | 58568585 | AAACGTCCACT | 102 |
| | | | TGCAGATTTTA | |
| | | | TGTAATTGGAA | |
| | | | GTTGGGG | |
| 6 | 8 | 56895765 | AGCAGGAAAAT | 106 |
| | | | ATATGCCCCAC | |
| | | | CTTCCCTTTCT | |
| | | | CTGACCC | |
| 7 | 1 | 121475300 | AGGAAGACTGC | 85 |
| | | | CTACTCCCACA | |
| | | | GGCCTGAAAGC | |
| | | | GCTCCAA | |
| 8 | X | 58563641 | AGCATTCGGGC | 67 |
| | | | CAGGGTTCACT | |
| | | | CAGGCTCAGGG | |
| | | | CACATTG | |
| 9 | 16 | 21525068 | GCATTTGGTGG | 38 |
| | | | TCTATAAGCAC | |
| | | | ACCCGCCCACA | |
| | | | CCAATCT | |
| 10 | 18 | 77932557 | CAAGACCAGCC | 26 |
| | | | TGAGGATGACT | |
| | | | GTCTCTTAGAG | |
| | | | GTGGAGA | |

Table 2 shows the target sequences identified in the ctDNA samples obtained from the blood of Subject 1. ctDNA samples are obtained from Subject 1 13 days before a tumor excision. As shown, each sequence contains at least partial viral genome sequence (underlined) and partial host genome sequence and forms a viral-host junction.

TABLE 2
Junction Data of Subject 1 Serum

| | Host | | | Accumu- |
|---|---|---|---|---|
| SEQ ID | Chromosome | Integration | Junction | lated |
| NO: | # | Position | Sequence | Reads |
| 11 | 17 | 22251295 | CACTCCTTTTG | 94 |
| | | | AGAGCGCAGTG | |
| | | | TTCAGGTGCAG | |
| | | | GGTCCCC | |
| 12 | 1 | 121360041 | ATCATCACAAA | 82 |
| | | | GTATTTTCTGA | |
| | | | GTCCTCTTATG | |
| | | | TAAGACC | |
| 13 | 1 | 13727 | AACAGAAAGAT | 68 |
| | | | TCGTCCCCAAA | |
| | | | TCCAATCTGTC | |
| | | | TTCCATC | |
| 14 | 8 | 56895765 | AGCAGGAAAAT | 62 |
| | | | ATATGCCCCAC | |
| | | | CTTCCCTTTCT | |
| | | | CTGCCCT | |
| 15 | 17 | 22247083 | GGTCTTACATA | 42 |
| | | | AGAGGACTCAG | |
| | | | AAAATACTTTG | |
| | | | TGATGAT | |
| 16 | 16 | 21525068 | GCATTTGGTGG | 31 |
| | | | TCTATAAGCAC | |
| | | | ACCCGCCCACA | |
| | | | CCAATCT | |
| 17 | 8 | 56895953 | ATCATCCTGGG | 16 |
| | | | CTTTCTGCACT | |
| | | | TCCCATAGGTA | |
| | | | ATCAAAG | |
| 18 | X | 58563641 | AGCATTCGGGC | 9 |
| | | | CAGGGTTCACT | |
| | | | CAGGCTCAGGG | |
| | | | CACATTG | |

As illustrated in Tables 1 and 2, at least SEQ 3 (in tumor sample) and SEQ 12 (in serum sample), SEQ 1 and SEQ 15, and SEQ 2 and SEQ 11 each pair have the same viral-host junction sequences. Similar patterns (including the relative amount of reads) of viral-host junction sequences identified in both tumor DNA and ctDNA indicate that chimera ctDNA in serum is derived from tumor DNA. By selectively enriching the ctDNAs carrying at least a portion of the viral genome in the serum, viral-host junctions are identified to provide tumor-specific information about the subject.

Subject 2

Table 3 shows the target sequences identified in the DNA samples obtained from Subject 2 tumor tissue. As shown, each sequence contains at least partial viral genome sequence (underlined) and partial host genome sequence and forms a viral-host junction.

TABLE 3
Junction Data of Subject 2 Tumor

| | Host | | | Accumu- |
|---|---|---|---|---|
| SEQ ID | Chromosome | Integration | Junction | lated |
| NO: | # | Position | Sequence | Reads |
| 19 | 3 | 111653312 | ATGAAGCTATT | 4183 |
| | | | TATAATAAAAC | |
| | | | AAACTTTATTA | |
| | | | AATCTAGTTTA | |
| | | | AATGCCTTACT | |
| | | | CTCTTTTTTGC | |
| | | | CTTCTGACTTC | |
| | | | TTTCCTTCTAT | |
| | | | TCGAGATCTCC | |
| | | | T | |
| 20 | 2 | 80278757 | TTTCATTGTTG | 3772 |
| | | | CTGTTTTTCAA | |
| | | | ATTGATTTTGG | |
| | | | GATCCAGCCTG | |
| | | | TTATTCTACTC | |
| | | | CCTTAACTTCA | |
| | | | TGGGATATGTA | |
| | | | ATTGGAAGTTG | |
| | | | GGGTACTTTAC | |
| | | | C | |
| 21 | 3 | 111653206 | TCTCCCTTTAG | 1269 |
| | | | ACTTCAAACAC | |
| | | | TTCAAAATATG | |
| | | | ACTTCACTACA | |
| | | | AAGCTTTATAG | |
| | | | AATGCCAGCCT | |
| | | | TCCACAGAGTA | |
| | | | TGTAAATAATG | |
| | | | CCTAGTTTTGA | |
| | | | A | |
| 22 | 2 | 80278655 | CCAGCACATTT | 752 |
| | | | GTCTATAAATT | |
| | | | TACATTCTTGG | |
| | | | ATATTAGCAAA | |
| | | | ATTGCAAACAG | |
| | | | ACCAATTTATG | |
| | | | CCTACAGCCTC | |
| | | | CTAGTACAAAG | |
| | | | ACCTTTAACCT | |
| | | | A | |
| 23 | 1 | 189879551 | TCCAGTGTTTG | 485 |
| | | | TGGGTTGAGCA | |
| | | | GTATTATTGCA | |
| | | | TGGCCCAGTGG | |
| | | | TGGTGGTTGAT | |
| | | | GTTCCTGGAAG | |
| | | | TAGAGGACAAA | |
| | | | CGGGCAACATA | |
| | | | CCTTGGTAGTC | |
| | | | C | |
| 24 | 1 | 189879474 | TGCAAGTGGTT | 174 |
| | | | GCAGTTCTTTT | |
| | | | GCTTTGCCACC | |
| | | | ACCACTGGGCC | |
| | | | ATGCAAAACCT | |
| | | | GCACGATTCCT | |
| | | | GCTCAAGGAAC | |
| | | | CTCTATGTTTC | |
| | | | CCTCTTGTTGC | |
| | | | T | |
| 25 | 20 | 60227034 | CAGGAGGAGGT | 169 |
| | | | GATGGACCCAC | |
| | | | TGGGTGGTGAA | |
| | | | GAACAGTTTCT | |
| | | | CTTCCAAAATT | |
| | | | ACTTCCCACCC | |
| | | | AGGTGGCCAGA | |
| | | | TTCATCAACTC | |
| | | | ACCCCAACACA | |
| | | | G | |
| 26 | 22 | 26941239 | ATCTGTAAAAT | 100 |
| | | | TGGGATCATCA | |
| | | | CACTTTCCTTT | |
| | | | TATTGGGGTTT | |
| | | | AAATGAATACC | |
| | | | CAAAGACAAAA | |
| | | | GAAAATTGGTA | |
| | | | ATAGAGGTAAA | |
| | | | AAGGGACTCAA | |
| | | | G | |
| 27 | 20 | 60227112 | TGGCCGAGGCC | 93 |
| | | | ATCTTCTAAAT | |
| | | | AAATGTGTGGA | |
| | | | AGAGAAACTGT | |
| | | | TCTTCAGTATT | |
| | | | TGGTGTCTTTT | |
| | | | GGAGTGTGGAT | |
| | | | TCGCACTCCTC | |
| | | | CCGCTTACAGA | |
| | | | C | |
| 28 | 5 | 1295309 | AGGACGGGTGC | 37 |
| | | | CCGGGTCCCCA | |
| | | | GTCCCTCCGCC | |
| | | | ACGTGGGAAGC | |
| | | | GCGGTCCAGAC | |
| | | | CAATTTATGCC | |
| | | | TACAGCCTCCT | |
| | | | AGTACAAAGAC | |
| | | | CTTTAACCTAA | |
| | | | T | |

Table 4 shows the target sequences identified in the ctDNA samples obtained from blood of Subject 2. Serum samples are obtained from Subject 2 at tumor excision. As shown, each sequence contains at least partial viral genome sequence (underlined) and partial host genome sequence and forms a viral-host junction.

TABLE 4
Junction Data of Subject 2 Serum

| | Host | | | Accumu- |
|---|---|---|---|---|
| SEQ ID | Chromosome | Integration | Junction | lated |
| NO: | # | Position | Sequence | Reads |
| 29 | 3 | 111653312 | ATGAAGCTATT | 3277 |
| | | | TATAATAAAAC | |
| | | | AAACTTTATTA | |
| | | | AATCTAGTTTA | |
| | | | AATGCCTTACT | |
| | | | CTCTTTTTTGC | |
| | | | CTTCTGACTTC | |
| | | | TTTCCTTCTAT | |
| | | | TCGAGATCTCC | |
| | | | T | |
| 30 | 20 | 60227034 | CAGGAGGAGGT | 642 |
| | | | GATGGACCCAC | |
| | | | TGGGTGGTGAA | |
| | | | GAACAGTTTCT | |
| | | | CTTCCAAAATT | |
| | | | ACTTCCCACCC | |
| | | | AGGTGGCCAGA | |
| | | | TTCATCAACTC | |
| | | | ACCCCAACACA | |
| | | | G | |
| 31 | 1 | 189879551 | TCCAGTGTTTG | 373 |
| | | | TGGGTTGAGCA | |
| | | | GTATTATTGCA | |
| | | | TGGCCCAGTGG | |
| | | | TGGTGGTTGAT | |
| | | | GTTCCTGGAAG | |
| | | | TAGAGGACAAA | |
| | | | CGGGCAACATA | |
| | | | CCTTGGTAGTC | |
| | | | C | |
| 32 | 2 | 50012582 | GTCCGTTGGTG | 372 |
| | | | GTGAACTGGGC | |
| | | | AAGATAATTGC | |
| | | | ATGGCCCAGTG | |
| | | | GTGGTGGTTGA | |
| | | | TGTTCCTGGAA | |
| | | | GTAGAGGACAA | |
| | | | ACGGGCAACAT | |
| | | | ACCTTGGTAGT | |
| | | | C | |
| 33 | 15 | 48344568 | AGATTGGTCTA | 237 |
| | | | TAATTTTCTTT | |
| | | | TACTATCTTCA | |
| | | | GTATTTGGTAT | |
| | | | CTTTGGGAGTG | |
| | | | TGGATTCGCAC | |
| | | | TCCTCCCGCTT | |
| | | | ACAGACCACCA | |
| | | | AATGCCCCTAT | |
| | | | C | |
| 34 | 2 | 80278757 | TTTCATTGTTG | 230 |
| | | | CTGTTTTTCAA | |
| | | | ATTGATTTTGG | |
| | | | GATCCAGCCTG | |
| | | | TTATTCTACTC | |
| | | | CCTTAACTTCA | |
| | | | TGGGATATGTA | |
| | | | ATTGGAAGTTG | |
| | | | GGGTACTTTAC | |
| | | | C | |
| 35 | 20 | 60227112 | TGGCCGAGGCC | 209 |
| | | | ATCTTCTAAAT | |
| | | | AAATGTGTGGA | |
| | | | AGAGAAACTGT | |
| | | | TCTTCAGTATT | |
| | | | TGGTGTCTTTT | |
| | | | GGAGTGTGGAT | |
| | | | TCGCACTCCTC | |
| | | | CCGCTTACAGA | |
| | | | C | |
| 36 | 1 | 189879474 | TGCAAGTGGTT | 205 |
| | | | GCAGTTCTTTT | |
| | | | GCTTTGCCACC | |
| | | | ACCACTGGGCC | |
| | | | ATGCAAAACCT | |
| | | | GCACGATTCCT | |
| | | | GCTCAAGGAAC | |
| | | | CTCTATGTTTC | |
| | | | CCTCTTGTTGC | |
| | | | T | |
| 37 | 2 | 50012660 | GTAAGCCATTG | 205 |
| | | | TGGCTTTCCTG | |
| | | | ACCAGCCCACC | |
| | | | ACCACTGGGCC | |
| | | | ATGCAAAACCT | |
| | | | GCACGATTCCT | |
| | | | GCTCAAGGAAC | |
| | | | CTCTATGTTTC | |
| | | | CCTCTTGTTGC | |
| | | | T | |
| 38 | 2 | 80278655 | CCAGCACATTT | 64 |
| | | | GTCTATAAATT | |
| | | | TACATTCTTGG | |
| | | | ATATTAGCAAA | |
| | | | ATTGCAAACAG | |
| | | | ACCAATTTATG | |
| | | | CCTACAGCCTC | |
| | | | CTAGTACAAAG | |
| | | | ACCTTTAACCT | |
| | | | A | |

As illustrated in Tables 3 and 4, at least SEQ 19 (in tumor sample) and SEQ 29 (in serum sample), SEQ 18 and SEQ 30, SEQ 23 and SEQ 21 both have the same viral-host junction sequences. Similar patterns of viral-host junction sequences identified in both tumor DNA and ctDNA show that chimera ctDNA in serum is derived from tumor DNA. By selectively enriching the target ctDNA in the serum, viral-host junctions are identified to provide tumor-specific information about the subject.

Subject 3

Table 5 shows the target sequences identified in the DNA samples obtained from Subject 3 tumor tissue. As shown, each sequence contains at least partial viral genome sequence (underlined) and partial host genome sequence and forms a viral-host junction.

TABLE 5
Junction Data of Subject 3 Tumor

| | Host | | | Accumu- |
|---|---|---|---|---|
| SEQ ID | Chromosome | Integration | Junction | lated |
| NO: | # | Position | Sequence | Reads |
| 39 | 5 | 1295930 | GGAAATGGAGC | 3024 |
| | | | CAGGCGCTCCT | |
| | | | GCTGGCCGCGC | |
| | | | ACCGGGCGCCT | |
| | | | CACACCAGAAC | |
| | | | ATCGCATCAGG | |
| | | | ACTCCTAGGAC | |
| | | | CCCTGCTCGTG | |
| | | | TTACAGGCGGG | |
| | | | G | |
| 40 | 8 | 111636420 | TCAAGCAGAAA | 635 |
| | | | AACCATGAAGA | |
| | | | TTTAAAAACTT | |
| | | | GTAAATATTTG | |
| | | | AATGTGGGCTC | |
| | | | CACCCCAACAG | |
| | | | TCCCCCGTGGG | |
| | | | GAGGGGTGAAC | |
| | | | CCTGGCCCGAA | |
| | | | T | |
| 41 | 14 | 52591737 | CTAAGGGACAC | 354 |
| | | | TACAGGAAACC | |
| | | | AGCCCCGAAGT | |
| | | | GATTTCTTTTG | |
| | | | AAATTCCAAAT | |
| | | | CTTTCTGTCCC | |
| | | | CAATCCCCTGG | |
| | | | GATTCTTCCCC | |
| | | | GATCATCAGTT | |
| | | | G | |
| 42 | 9 | 138857330 | CCTCGAAGCCT | 190 |
| | | | GTGCCAACCTA | |
| | | | GCCCATTCCTC | |
| | | | AGGCTCAGGGC | |
| | | | CTCCTCACATC | |
| | | | TGTGCCAGCAG | |
| | | | CTCCTCCTCCT | |
| | | | GCCTCCACCAA | |
| | | | TCGGCAGTCAG | |
| | | | G | |
| 43 | 1 | 68549419 | CATTGTTACTG | 188 |
| | | | TGATATGCTAT | |
| | | | AATTATTCTCA | |
| | | | CCTTATGTGTC | |
| | | | CAAGGAATACT | |
| | | | AACATTGAGAT | |
| | | | TCCCGAGATTG | |
| | | | AGATCTTCTGC | |
| | | | GACGCGGCGAT | |
| | | | T | |
| 44 | 9 | 31455679 | ATGGAGAATAC | 172 |
| | | | AGCACATTATT | |
| | | | AGGAGTAAGTT | |
| | | | TCCTTAAACAC | |
| | | | ATTTTGATTTT | |
| | | | TTGTACAATAT | |
| | | | GTTCCTGTGGC | |
| | | | AATGTGCCCCA | |
| | | | ACTCCCAATTA | |
| | | | C | |
| 45 | 17 | 71434403 | TTTGCCACCTT | 138 |
| | | | CCTGCCACTTT | |
| | | | GTAGATGCAAG | |
| | | | ATCTTGGGCAA | |
| | | | GTTCCCGTGGG | |
| | | | CGTTCACGGTG | |
| | | | GTTTCCATGCG | |
| | | | ACGTGCAGAGG | |
| | | | TGAAGCGAAGT | |
| | | | G | |
| 46 | 12 | 126230889 | CAGTGGAAACA | 135 |
| | | | AAGCCACTGGG | |
| | | | AAGTTCAAACT | |
| | | | GAGAGAAGCCC | |
| | | | ACCACAAGTCT | |
| | | | AGACTCTGTGG | |
| | | | TATTGTGAGGA | |
| | | | TTTTTGTCAAC | |
| | | | AAGAAAAACCC | |
| | | | C | |
| 47 | X | 35911295 | AGTATATCATC | 124 |
| | | | AGTTATTTTTC | |
| | | | AAGGTTTTCTA | |
| | | | AGTAAACAGTT | |
| | | | TCTCAACCTTT | |
| | | | ACCCCGTTGCT | |
| | | | CGGCAACGGCC | |
| | | | TGGTCTGTGCC | |
| | | | AAGTGTTTGCT | |
| | | | G | |
| 48 | 10 | 75397400 | TCAGGGAGGGG | 58 |
| | | | ATGTTGACTGC | |
| | | | ATTTTGGAGGT | |
| | | | TCAGGGCCTAC | |
| | | | TAACAACTGTG | |
| | | | CCAGCAGCTCC | |
| | | | TCCTCCTGCCT | |
| | | | CCACCAATCGG | |
| | | | CAGTCAGGAAG | |
| | | | G | |

Table 6 shows the target sequences identified in the ctDNA samples obtained from blood of Subject 3. Serum samples are obtained from Subject 3 at tumor excision. As shown, each sequence contains at least partial viral genome sequence (underlined) and partial host genome sequence and forms a viral-host junction.

TABLE 6
Junction Data of Subject 3 Serum

| | Host | | | Accumu- |
|---|---|---|---|---|
| SEQ ID | Chromosome | Integration | Junction | lated |
| NO: | # | Position | Sequence | Reads |
| 49 | 5 | 1295930 | GGAAATGGAGC | 153 |
| | | | CAGGCGCTCCT | |
| | | | GCTGGCCGCGC | |
| | | | ACCGGGCGCCT | |
| | | | CACACCAGAAC | |
| | | | ATCGCATCAGG | |
| | | | ACTCCTAGGAC | |
| | | | CCCTGCTCGTG | |
| | | | TTACAGGCGGG | |
| | | | G | |
| 50 | 8 | 111636420 | TCAAGCAGAAA | 52 |
| | | | AACCATGAAGA | |
| | | | TTTAAAAACTT | |
| | | | GTAAATATTTG | |
| | | | AATGTGGGCTC | |
| | | | CACCCCAACAG | |
| | | | TCCCCCGTGGG | |
| | | | GAGGGGTGAAC | |
| | | | CCTGGCCCGAA | |
| | | | T | |
| 51 | 21 | 47565536 | CCCGGGACCGA | 27 |
| | | | CCCCAGGAAGA | |
| | | | GCCAGGGGCCC | |
| | | | GGGTGATCCCT | |
| | | | GCGGGGGTCTG | |
| | | | GCTTTCAGTTA | |
| | | | TATGGATGATG | |
| | | | TGGTATTGGGG | |
| | | | GCCAAGTCTGT | |
| | | | A | |
| 52 | 21 | 28573066 | AATGAAAATCT | 25 |
| | | | CATTGATTTTT | |
| | | | CACTTATAGGT | |
| | | | TTTACCTTAGA | |
| | | | GCTCCTCCTCT | |
| | | | GCCTAATCATC | |
| | | | TCATGTTCATG | |
| | | | TCCTACTGTTC | |
| | | | AAGCCTCCAAG | |
| | | | C | |
| 53 | 7 | 87842849 | AGAATTGATAC | 24 |
| | | | CTAAGCTGAGC | |
| | | | AGAAATGAGGC | |
| | | | CGACCATGAAG | |
| | | | TGAGTGCCTAA | |
| | | | TCATCTCATGT | |
| | | | TCATGTCCTAC | |
| | | | TGTTCAAGCCT | |
| | | | CCAAGCTGTGC | |
| | | | C | |
| 54 | 7 | 148503201 | CGTAGGAAAGA | 19 |
| | | | CAAGGTGGCAT | |
| | | | TGATGGAAAGC | |
| | | | AGTAGTTTTTG | |
| | | | AGCCCTTCGCA | |
| | | | GACGAAGGTCT | |
| | | | CAATCGCCGCG | |
| | | | TCGCAGAAGAT | |
| | | | CTCAATCTCGG | |
| | | | G | |
| 55 | 1 | 162277132 | TTAAAAAGGAG | 16 |
| | | | TTTTGTTTGTT | |
| | | | AGTCTATTCAC | |
| | | | TCATTTCAAGG | |
| | | | AACATAGAAGA | |
| | | | AGAACTCCCTC | |
| | | | GCCTCGCAGAC | |
| | | | GAAGGTCTCAA | |
| | | | TCGCCGCGTCG | |
| | | | C | |
| 56 | 12 | 125048731 | CAGTTCCCTGG | 15 |
| | | | CTCCAAGCTCC | |
| | | | CTCAAAAGATG | |
| | | | CCCAGCTGGCC | |
| | | | TTTCCCAAAGG | |
| | | | CCTTGTAAGTT | |
| | | | GGCGAGAAAGT | |
| | | | AAAAGCCTGTT | |
| | | | TTGCTTGTATA | |
| | | | C | |
| 57 | 7 | 30412226 | ACATGCCCTTC | 13 |
| | | | ACTTCAGCCTG | |
| | | | ATGCTCCTGGC | |
| | | | ATAAGCTCAGC | |
| | | | AATTTTGGAGT | |
| | | | GCGAATCCACA | |
| | | | CTCCAAAAGAC | |
| | | | ACCAAATATTC | |
| | | | AAGAACAGTTT | |
| | | | C | |
| 58 | 13 | 84505952 | AATTTCCCCTG | 13 |
| | | | AATAGCTGCAG | |
| | | | TACTCACAGAC | |
| | | | ACACTGGATGC | |
| | | | TACTCACCTCT | |
| | | | GCCTAATCATC | |
| | | | TCATGTTCATG | |
| | | | TCCTACTGTTC | |
| | | | AAGCCTCCAAG | |
| | | | C | |

As illustrated in Tables 5 and 6, similar patterns of viral-host junction sequences identified in both tumor DNA and ctDNA show that ctDNA in serum is derived from tumor DNA. By selectively enriching the target ctDNA in the serum, viral-host junctions are identified to provide tumor-specific information about the subject.

3.2 Tumor-Specific Viral-Host Junctions

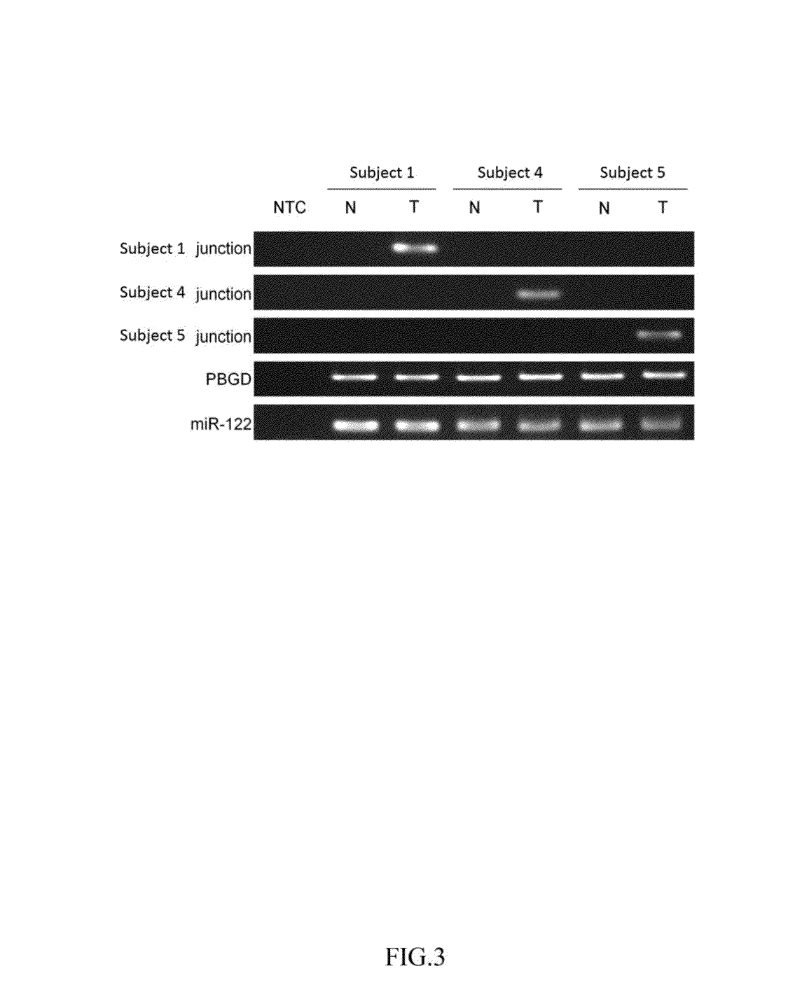

In FIG. 3, genomic DNA of Subject 1, Subject 4 and Subject 5 are processed and analyzed by polymerase chain reaction (PCR) and quantitative PCR. Genomic DNA (gDNA) from tumor tissues and non-tumor tissues is obtained. One chimera DNA sequence in tumor gDNA is identified and selected in each subject to serve as a marker to conduct the tests. Specifically, the chimera DNA sequence of Subject 1 used in this analysis is GGTCTTACATAAGAGGACTCAGAAAATACTTTGTGATGAT (viral genome sequence underlined), Subject 4 ACTTCAAAGACTGTGTGTTTCTAATTATTTTGGGGGACAT, and Subject 5 GTAGGCATAAATTGGTCTGTACCTCACTTCCCTGCTTTCC. The presence of the three specific viral-host junctions is determined in the tumor gDNA (T) and non-tumor gDNA (N). Porphobilinogen deaminase (PBGD) and miR-122 are used as internal control. No-template control (NTC) is also included. As illustrated in FIG. 3, the specific viral-host junction of Subject 1 is only present in tumor gDNA (T) but not in non-tumor gDNA (N). Same patterns are observed in Subject 4 and Subject 5, indicating that the identified viral-host junctions are tumor-specific and can be used as tumor-specific biomarkers.

3.3 Tumor Development and Viral-Host Junction Amount

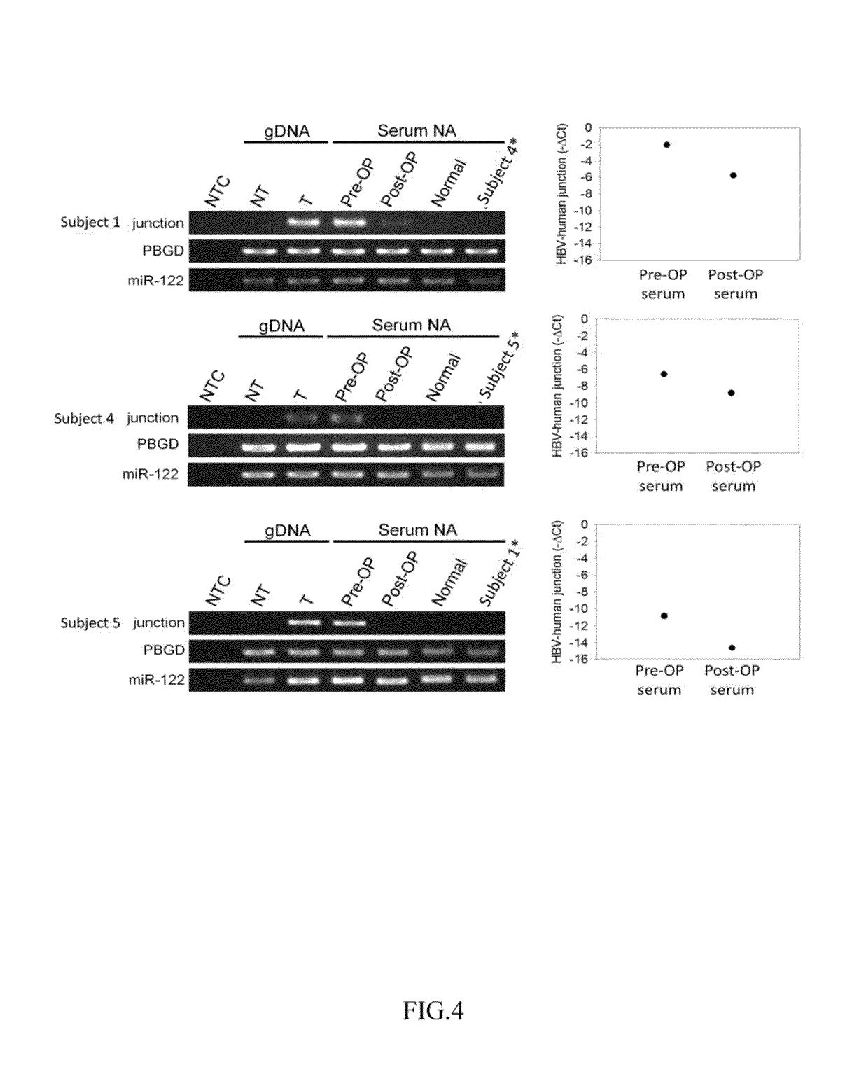

FIG. 4 shows the relationships between tumor size and the amount of specific viral-host junction sequence. Junction sequences used in FIG. 4 for each subject are the same as in FIG. 3. Serial blood samples of each subject are obtained at least at pre-operation and post-operation stages. Referring to FIG. 4, gDNA refers to genomic DNA, NTC refers to no-template control, NT refers to gDNA from non-tumor tissue, T refers to gDNA from tumor tissue, Serum NA refers to DNA obtained from serum, Pre-OP refers to serum DNA obtained at pre-operation stage, Post-OP refers to serum DNA obtained at post-operation stage, Subject 1* refers to serum DNA obtained from Subject 1 and is used in Subject 5 experiment, Subject 4* refers to serum DNA obtained from Subject 4 and is used in Subject 1 experiment, Subject 5* refers to serum DNA obtained from Subject 5 and is used in Subject 4 experiment and Normal refers to serum DNA obtained from a non-patient subject. Serum samples of Subject 1 are obtained at two time points, 13 days before tumor resection (operation) and 19 days after operation. Serum samples of Subject 4 are obtained 33 days before operation and 30 days after operation. Serum samples of Subject 5 are obtained 24 days before operation and 26 days after operation. Serum samples of non-patient subject (Normal) are also included as a control in FIG. 4. As shown in FIG. 4 left panel, the specific viral-host junction of each subject is only present in tumor gDNA and Pre-OP. In addition, the specific viral-host junction of Subject 1 is only present in Subject 1 DNA samples but not in Subject 4 DNA samples, suggesting that the viral-host junction identified is subject-specific. Referring now to the right panel of FIG. 4, the specific viral-host junction in the serum of Subject 1 is detected with relatively large amount in Pre-OP serum while the amount decreases sharply in Post-OP serum. The amount of the specific viral-host junction in Pre-OP serum and Post-OP serum is determined by qPCR and presented in the right panel of FIG. 4. In Subject 1, the amount of specific viral-host junction in Post-OP serum decreases by about 32-fold compared to in Pre-OP serum. Same patterns are observed in Subject 4 and Subject 5, showing that the viral-host junctions or the amount of junctions are tumor-specific, subject-specific, detectable in serum, reflective of tumor, and corresponsive to the tumor size changes, such as a decrease in size after an operation.

It is to be noted that by using the approach described in the present invention, mutated p53 or beta-catenin genes cannot be detected in the ctDNAs despite the mutations are identified in the tumor tissues (data not shown). The result shows that by using the method of present invention, tumor specific viral-host junctions (viral genome sequence insertion into host genome), and not conventional somatic mutations, are selectively enriched and obtained to provide cancer/tumor information.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

What is claimed is:

1. A substantially cell-free nucleic acid isolated from the circulation of a subject, comprising:
at least one sequence derived from host genome;
at least one sequence derived from hepatitis B viral genome;
wherein the at least one sequence derived from host genome and the at least one sequence derived from hepatitis B viral genome form a chimera junction;
wherein the chimera junction is obtained from substantially cell-free nucleic acids; and
wherein the chimera junction is indicative of disease status.

2. The nucleic acid of claim 1, wherein the chimera junction is separated from non-chimeric nucleic acids by using at least one probe derived from non-host sequence complementary to the at least one sequence derived from hepatitis B viral genome.

3. The nucleic acid of claim 2, wherein the disease status is a tumor status; and
wherein the chimera junction is derived from the tumor.

4. The nucleic acid of claim 3, wherein the non-host sequence is hepatitis B viral genome.

5. The nucleic acid of claim 3, wherein the tumor is a hepatocelluar carcinoma induced by hepatitis B virus.

6. A method of identifying circulating cell-free DNA from a subject infected with hepatitis B virus comprising:
determining presence, absence, or amount of at least one viral-host junction in the circulating cell-free DNA;
wherein the at least one viral-host junction is selectively enriched by contacting the circulating cell-free DNA with at least one probe complementary to at least one sequence derived from hepatitis B viral genome and capturing the circulating cell-free DNA hybridized with the at least one probe; and
wherein the at least one viral-host junction is a biomarker indicative of hepatitis B virus-related tumor status.

7. The method of claim 6, wherein the at least one viral-host junction comprises at least one hepatitis viral B genomic sequence and at least one non-viral host genomic sequence.

8. A method of monitoring a tumor in a subject, comprising:
contacting circulating cell-free DNA from the subject with at least one probe complementary to at least one sequence derived from hepatitis B viral genome;
capturing the circulating cell-free DNA hybridized with the at least one probe;
determining the presence, absence, or amount of at least one viral-host junction in the circulating cell-free DNA.

9. The method of claim 8, wherein the at least one viral-host junction identified in different samples obtained at different time points of the subject is indicative of the tumor status.

10. The method of claim 9, wherein the tumor is related to infection of the subject by hepatitis B virus.

11. The method of claim 10, wherein the tumor is a hepatocelluar carcinoma induced by hepatitis B virus.

12. The method of claim 11, wherein the different time points are selected from a cancerous condition, a pre-treatment condition, a post-treatment condition, a recurrence condition of the subject, and any combination thereof.

13. The method of claim 12, wherein changes in the amount of the at least one viral-host junction at different time points are indicative of the tumor development of the subject from one condition to another.

14. The method of claim 13, wherein increases in the amount of the at least one viral-host junction in the circulating cell-free DNA of the subject are indicative of the tumor development from the post-treatment condition to a recurrence condition and decreases in the amount of the at least one viral-host junction in the circulating cell-free DNA of the subject are indicative of the tumor development from the pre-treatment condition to a post-treatment condition of the subject.

15. The method of claim 13, wherein increases in the amount of the at least one viral-host junction in the circulating cell-free DNA of the subject in the cancerous condition are indicative of growth of a tumor and decreases in the amount of the at least one viral-host junction in the circulating cell-free DNA of the subject in the cancerous condition are indicative of shrinkage of the tumor.

16. A biomarker in a subject, comprising:
a nucleic acid comprising at least a portion of a host sequence from a host genome and at least a portion of a viral sequence from a viral genome;
a viral-host junction formed by the conjunction of the at least a portion of the host sequence from the host genome and the at least a portion of the viral sequence from the viral genome;
wherein the nucleic acid is obtained from circulating cell-free DNA by contacting the circulating cell-free DNA with polynucleotides complementary to the at least a portion of the viral sequence and capturing the nucleic acids hybridized with the polynucleotides.

17. The biomarker of claim 16, wherein the host genome is a human genome and the viral genome is a hepatitis B virus genome.

18. The biomarker of claim 17, wherein the biomarker is a tumor-specific biomarker.

19. A method of diagnosing a disease in a subject infected with hepatitis B virus, comprising:
detecting one or more circulatory cell-free DNAs from a subject, wherein the one or more circulatory cell-free DNAs comprise at least one sequence derived from non-host hepatitis B viral genome and at least one sequence derived from host genome.

20. The method of claim 19, wherein the disease is a cancer caused by chronic infection of hepatitis B virus.
